(19) United States
(12) Patent Application Publication
(10) Pub. No.: US 2017/0055977 A1
(43) Pub. Date: Mar. 2, 2017

(54) MULTIPLE-NEEDLE SUTURING ASSEMBLY

(71) Applicant: Nir Altman, Kfar Etzion (IL)

(72) Inventor: Nir Altman, Kfar Etzion (IL)

(21) Appl. No.: 14/833,247

(22) Filed: Aug. 24, 2015

(51) Int. Cl. A61B 17/04

(52) U.S. Cl. CPC A61B 17/0469; A61B 2017/0472

(57) ABSTRACT

A suturing assembly includes a suture passing member formed with a lumen in which are disposed a suture holding element and a first puncture element. Both the first puncture element and the suture holding element include suture receiving portions. A second puncture element includes a suture receiving portion. The first puncture element and the suture holding element have a retracted position and a deployed position. In the deployed position, the distal end of the first puncture element and/or suture holding element bends and passes over the distal end of the second puncture element, and a space is formed between the first puncture element and the suture holding element. During movement from the deployed position back to the retracted position, the suture receiving portion of the first puncture element and/or suture holding element moves adjacent the suture receiving portion of the second puncture element.

### FIELD OF THE INVENTION

The present invention relates generally to suturing devices and methods, such as for the percutaneous closure of body lumens and tissues by means of sutures, and particularly to a suturing assembly with a bendable needle.

### BACKGROUND OF THE INVENTION

Many suturing devices are known in the art that puncture the skin with needles and percutaneously deliver sutures to the site. However, in endoscopic procedures, where access to the work site is not readily available, the surgeon must use auxiliary devices to be able to grasp the suture strands and pass them through desired tissue.

U.S. Pat. No. 7,842,050 describes a suture passing device which can advance, retract, and/or snare suture strands. The device incorporates superelastic puncturing components that exhibit superelastic properties such that they can be compressed into a straightening tube or integrated lumen of a device having a substantially smaller profile during deployment. The superelastic puncturing components of the suture passing devices return towards their preformed, resting, enlarged shape upon removal of the compressive forces required to reduce their profiles thereby enabling the advancement or retraction of one or more sutures through soft tissue despite limited access to the soft tissue, characteristic of less invasive procedures. Another requirement of the superelastic puncturing components of the suture passing devices is an integral keyhole, eyelet opening or crochet hook capable of advancing and/or retracting suture strands coincident with manipulations of the puncturing device.

### SUMMARY OF THE INVENTION

The present invention seeks to provide an improved suturing assembly, as described in more detail further below.

The assembly and methods of the present invention can be used to close a tissue puncture site with suture thread, such as a puncture site through the wall of a body lumen, or a percutaneous vascular puncture site at the distal end of a tissue tract. The assembly and methods can achieve closure wholly within the tissue tract leading to a puncture site and can be manipulated entirely from the portion of the device lying outside of the tissue tract. The present invention may be used in various medical procedures, such as but not limited to, laparoscopic procedures, endoscopic procedures, arthroscopic procedures, and the like. There is thus provided in accordance with an embodiment of the present invention a suturing assembly a suture passing member formed with a lumen in which are disposed a suture holding element and a first puncture element formed with a sharp distal end for puncturing tissue, both the first puncture element and the suture holding element including suture receiving portions, and a second puncture element including a suture receiving portion and a sharp distal end for puncturing tissue, wherein the first puncture element and the suture holding element have a retracted position and a deployed position, wherein in the retracted position, a distal end of the suture holding element does not protrude beyond the sharp distal end of the first puncture element, and in the deployed position:

a. the first puncture element and the suture holding element are positioned further away from a distal end of the lumen than in the retracted position,

b. the distal end of one of the first puncture element and the suture holding element bends and passes over the distal end of the second puncture element,

c. a space is formed between the first puncture element and the suture holding element and the second puncture element is positioned opposite the space, and

d. during movement of the first puncture element and the suture holding element from the deployed position back to the retracted position, the suture receiving portion of the one of the first puncture element and the suture holding element that passes over the distal end of the second puncture element, moves adjacent the suture receiving portion of the second puncture element.

In accordance with an embodiment of the present invention a suture is initially disposed in the suture receiving portions of the first puncture element and the suture holding element, and the movement of the first puncture element and the suture holding element from the deployed position back to the retracted position causes the suture to move against the second puncture element and become disengaged from the suture receiving portions of the first puncture element and the suture holding element and to pass into the suture receiving portion of the second puncture element.

In accordance with an embodiment of the present invention, in the retracted position, the suture receiving portions of the first puncture element and the suture holding element are aligned and flush with each other.

In accordance with an embodiment of the present invention the distal ends of both of the first puncture element and the suture holding element bend and pass over the distal end of the second puncture element.

In accordance with an embodiment of the present invention the distal end of the one of the first puncture element and the suture holding element bends to a curved position to pass over the distal end of the second puncture element.

In accordance with an embodiment of the present invention the one of the first puncture element and the suture holding element that bends to the curved position is made of a shape memory material and curves due to shape memory properties of the material.

In accordance with an embodiment of the present invention the suture receiving portion of the second puncture element includes a longitudinal channel with a distal closed end and a proximal closed end and a side opening between the distal and proximal closed ends, the side opening being formed through an outer contour of the second puncture element, and wherein the outer contour includes a distal slanted portion, which is proximal to the sharp distal end of the second puncture element and which slants proximally inwards towards the side opening, and a proximal slanted portion which is proximal to the distal slanted portion and which slants distally inwards towards the side opening.

In accordance with an embodiment of the present invention the proximal closed end slants proximally towards the proximal slanted portion.

In accordance with an embodiment of the present invention a handle is connected to the first puncture element and the suture holding element, and a biasing device is operative to urge the first puncture element and the suture holding element from the deployed position back to the retracted position.

There is also provided in accordance with an embodiment of the present invention a method for suturing including providing a first puncture element including a sharp distal end for puncturing tissue, providing a second puncture element including a sharp distal end for puncturing tissue, the distal ends of the first and second puncture elements being spaced from each other by a gap, providing suture thread disposed along a portion of the first puncture element, wherein the suture thread is arranged to be grabbed at the distal end of the first puncture element, providing a suture grabber positioned at the distal end of the second puncture element, puncturing through a tissue wall with the first puncture element so that the suture thread passes from a near side of the tissue wall to a far side of the tissue wall, puncturing through the tissue wall with the second puncture element from the near side of the tissue wall to the far side of the tissue wall, and grabbing the suture thread with the suture grabber at the distal end of the first puncture element at the far side of the tissue wall, bringing the suture thread across the gap and moving the suture thread proximally away from the distal end of the second puncture element back through to the near side of the tissue wall. Afterwards, the suture thread may be secured to form a stitch.

### BRIEF DESCRIPTION OF THE DRAWINGS

The present invention will be understood and appreciated more fully from the following detailed description, taken in conjunction with the drawings in which:

FIG. 1 is a simplified pictorial illustration of a suturing assembly, constructed and operative in accordance with a non-limiting embodiment of the present invention;

FIG. 2 is a simplified pictorial illustration of the suturing assembly of FIG. 1, showing a biasing device which urges the first puncture element and the suture holding element from the deployed position back to the retracted position;

FIGS. 3A and 3B are simplified pictorial illustrations of the suturing assembly of FIG. 1, showing the suture holding element and the first and second puncture elements, respectively with and without a suture received in the suture receiving portions;

FIG. 3C is an enlarged pictorial illustration of the first puncture element and the suture holding element, showing a lower rim for leaning on the distal end of the lumen of the suture passing member; and

FIGS. 4A, 4B, 4C and 4D are simplified illustrations of progressive steps in using the suturing assembly of FIG. 1 after puncturing a suture site, in accordance with a non-limiting embodiment of the present invention, respectively at initial deployment of the first puncture element and the suture holding element, further deployment, bending and passing over the second puncture element, and the final deployed position plus (in broken lines) movement from the deployed position back to the retracted position.

### DETAILED DESCRIPTION OF EMBODIMENTS

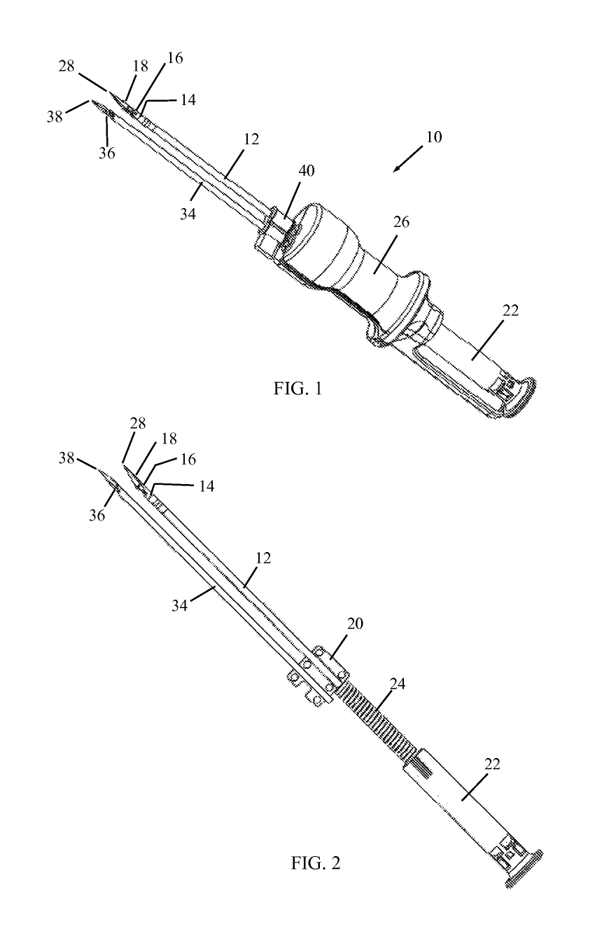

Reference is now made to FIG. 1, which illustrates a suturing assembly 10, constructed and operative in accordance with a non-limiting embodiment of the present invention.

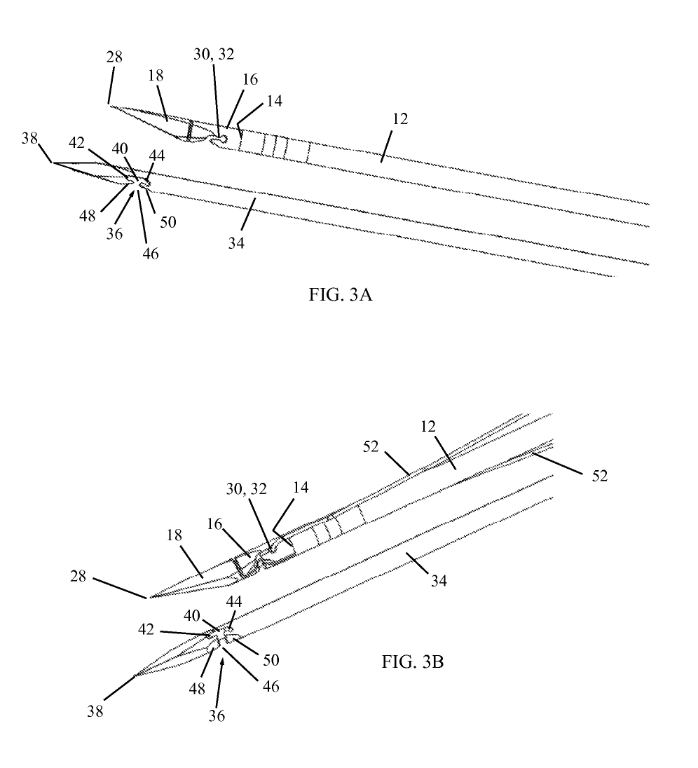

The suturing assembly 10 includes a suture passing member 12, which is formed with a lumen 14. A suture holding element 16 and a first puncture element 18 are disposed in lumen 14. As seen in FIG. 2, the proximal ends of suture passing member 12, that is, the proximal ends of suture holding element 16 and first puncture element 18, pass through or are attached to a mounting member 20 and are secured to a pushing member 22. A biasing device 24, such as a coil spring, is positioned between mounting member 20 and pushing member 22. The action of pressing on the pushing member 22 compresses biasing device 24 and advances the distal ends of suture holding element 16 and first puncture element 18 further away from the distal end of lumen 14. As seen in FIG. 1, a handle 26 is provided that encloses the mounting member and biasing device.

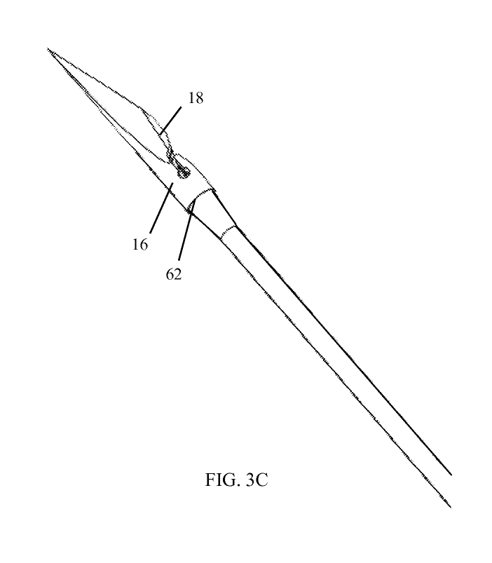

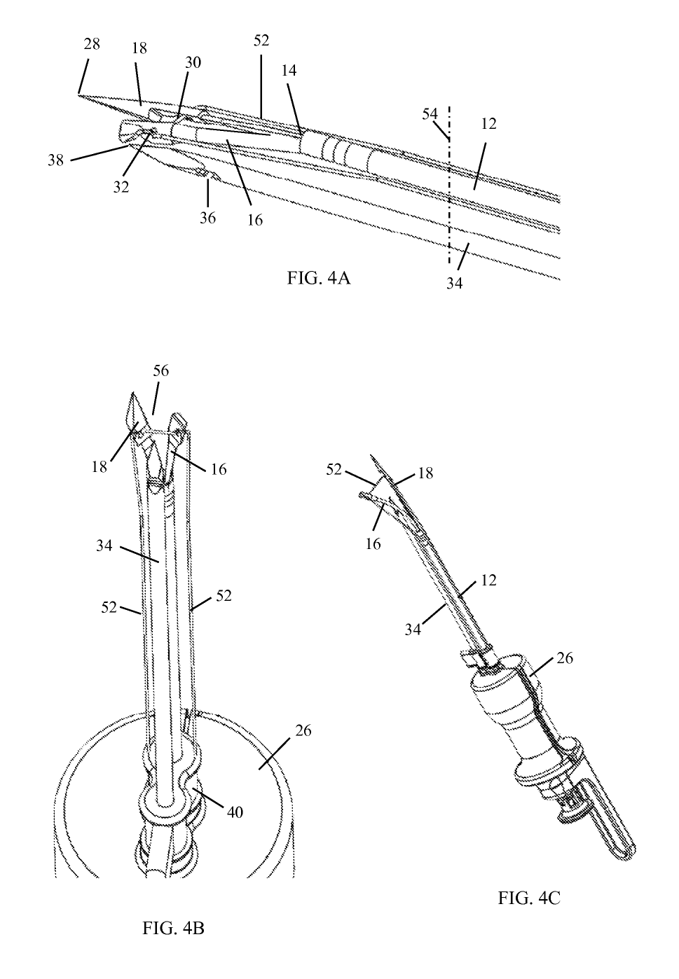

First puncture element 18 is formed with a sharp distal end 28 for puncturing tissue. As seen in FIGS. 3A and 3B, both first puncture element 18 and suture holding element 16 include suture receiving portions 30 and 32, respectively.

A second puncture element 34 includes a suture receiving portion 36 and a sharp distal end 38 for puncturing tissue. A spacer element 40 may be provided for maintaining a space between suture passing member 12 and second puncture element 34. The spacer element 40 may be used to maintain a space between any puncturing elements, such as between the first and second puncture elements, even without a tube such as member 12.

As seen in FIGS. 3A and 3B, the suture receiving portion 36 of second puncture element 34 includes a longitudinal channel 40 with a distal closed end 42 and a proximal closed end 44 and a side opening 46 between the distal and proximal closed ends 42 and 44. The side opening 46 is formed through an outer contour of second puncture element 34. This outer contour includes a distal slanted portion 48, which is proximal to the sharp distal end 38 and which slants proximally inwards towards side opening 46, and a proximal slanted portion 50 which is proximal to the distal slanted portion 48 and which slants distally inwards towards side opening 46. The proximal closed end 44 may slant proximally towards the proximal slanted portion 50, which may be helpful in ensnaring and holding suture thread during the stitching process. The slanted portions 48 and 50 help in guiding the suture thread into the longitudinal channel 40 of suture receiving portion 36 during the stitching process.

In FIG. 3C, it is seen that first puncture element 18 and/or suture holding element 16 are formed with a lower rim 62 for leaning on the distal end of lumen of the suture passing member tube (not shown here). If there were no such rim, the first puncture element 18 and/or suture holding element 16 would behave as a long cantilevered slender element coming out of the suture passing member tube and would be quite flexible with less strength to pierce through tissue. By providing rim 62 which is supported on the distal end of the lumen, the cantilevered distance is greatly reduced and this greatly increases the stiffness and strength of the first puncture element 18 and/or suture holding element 16 for piercing through tissue.

FIGS. 1-3B illustrate first puncture element 18 and suture holding element 16 in the retracted position. The distal end of suture holding element 16 does not protrude beyond the sharp distal end 28 of first puncture element 18. Prior to use, a suture 52 (FIG. 3B) is initially disposed in the suture receiving portions 30 and 32 of first puncture element 18 and suture holding element 16. In the illustrated embodiment, in the retracted position, the suture receiving portions 30 and 32 are aligned and flush with each other. Alternatively, the suture receiving portions 30 and 32 can be staggered or offset.

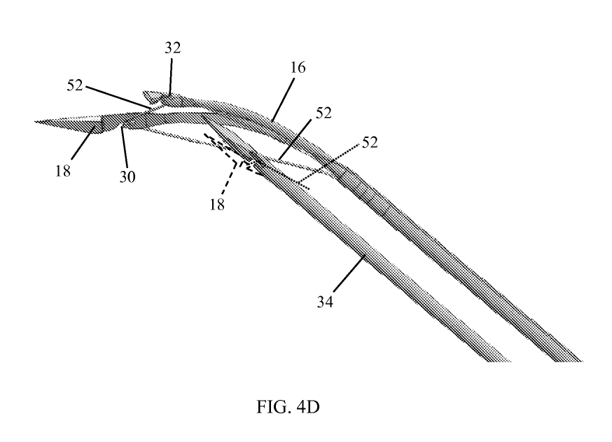

Reference is now made to FIGS. 4A, 4B, 4C and 4D, which illustrate progressive steps in using suturing assembly 10, in accordance with a non-limiting embodiment of the present invention.

In operation of suturing assembly 10, the sharp distal ends 28 and 38 of first and second puncture elements 18 and 34, respectively, are first pushed through a tissue wall at a suture site 52 (indicated by broken lines in FIG. 4A). In the illustrated embodiment, the first and second puncture elements puncture through the tissue wall basically at the same time, but it is possible to construct the assembly so they puncture the tissue wall one after the other. It is also possible to construct the assembly so that the puncture elements are not on a single tool but are on separate tools.

In FIG. 4A, first puncture element 18 and suture holding element 18 are positioned further away from the distal end of lumen 14 than in the retracted position (by pushing the pushing member 22, as described above).

In FIG. 4B, a space 56 is formed between first puncture element 18 and suture holding element 16, and second puncture element 34 is positioned opposite the space 56.

In FIG. 4C, first puncture element 18 and suture holding element 16 are moved further distally out of lumen 14, so that the distal end of first puncture element 18 and/or suture holding element 16 bends and passes over the distal end of second puncture element 34. In accordance with an embodiment of the present invention, the distal end of first puncture element 18 and/or suture holding element 16 bends to a curved position because they are made of a shape memory material (e.g., nitinol). First puncture element 18 and suture holding element 16 can be separate elements, but in a preferred embodiment, a single element has a split or bifurcation to form the first puncture element 18 and suture holding element 16. Alternatively, the first puncture element 18 and suture holding element 16 can each be formed with a hinge which is initially retracted in the lumen 14, but when deployed out of the lumen, the distal end articulates and bends about the hinge to the desired position.

FIG. 4D illustrates the final deployed position in solid lines and illustrates in broken lines the movement from the deployed position back to the retracted position.

During movement of first puncture element 18 and suture holding element 16 from the deployed position back to the retracted position, the suture receiving portion 30 and/or 32 of first puncture element 18 and/or suture holding element 16 moves adjacent the suture receiving portion 36 of second puncture element 34. The movement of first puncture element 18 and suture holding element 16 from the deployed position back to the retracted position causes suture 52 to move against second puncture element 34 and become disengaged from the suture receiving portions 30 and 32 and to pass into the suture receiving portion 36 of second puncture element 34.

After the suture 52 has been received in suture receiving portion 36 of second puncture element 34, first puncture element 18 and suture holding element 16 are fully retracted back into lumen 14 and the assembly is pulled back to the near side of the suture site 52. Afterwards, the suture 52 may be secured to form a stitch.

What is claimed is:

1. A suturing assembly comprising:
a suture passing member formed with a lumen in which are disposed a suture holding element and a first puncture element formed with a sharp distal end for puncturing tissue, both said first puncture element and said suture holding element comprising suture receiving portions; and
a second puncture element comprising a suture receiving portion and a sharp distal end for puncturing tissue;
wherein said first puncture element and said suture holding element have a retracted position and a deployed position,
wherein in the retracted position, a distal end of said suture holding element does not protrude beyond the sharp distal end of said first puncture element;
and in the deployed position:
a. said first puncture element and said suture holding element are positioned further away from a distal end of said lumen than in the retracted position,
b. the distal end of one of said first puncture element and said suture holding element bends and passes over the distal end of said second puncture element,
c. a space is formed between said first puncture element and said suture holding element and said second puncture element is positioned opposite said space, and
d. during movement of said first puncture element and said suture holding element from the deployed position back to the retracted position, the suture receiving portion of the one of said first puncture element and said suture holding element that passes over the distal end of said second puncture element, moves adjacent the suture receiving portion of said second puncture element.

2. The suturing assembly according to claim 1, further comprising a suture initially disposed in the suture receiving portions of said first puncture element and said suture holding element, and wherein the movement of said first puncture element and said suture holding element from the deployed position back to the retracted position causes said suture to move against said second puncture element and become disengaged from the suture receiving portions of said first puncture element and said suture holding element and to pass into the suture receiving portion of said second puncture element.

3. The suturing assembly according to claim 1, wherein in the retracted position, the suture receiving portions of said first puncture element and said suture holding element are aligned and flush with each other.

4. The suturing assembly according to claim 1, wherein the distal ends of both of said first puncture element and said suture holding element bend and pass over the distal end of said second puncture element.

5. The suturing assembly according to claim 1, wherein the distal end of the one of said first puncture element and said suture holding element bends to a curved position to pass over the distal end of said second puncture element.

6. The suturing assembly according to claim 5, wherein the one of said first puncture element and said suture holding element that bends to the curved position is made of a shape memory material and curves due to shape memory properties of said material.

7. The suturing assembly according to claim 1, wherein the suture receiving portion of said second puncture element comprises a longitudinal channel with a distal closed end and a proximal closed end and a side opening between the distal and proximal closed ends, said side opening being formed through an outer contour of said second puncture element, and wherein said outer contour comprises a distal slanted portion, which is proximal to the sharp distal end of said second puncture element and which slants proximally inwards towards said side opening, and a proximal slanted portion which is proximal to the distal slanted portion and which slants distally inwards towards said side opening.

8. The suturing assembly according to claim 7, wherein the proximal closed end slants proximally towards said proximal slanted portion.

9. The suturing assembly according to claim 1, further comprising a handle connected to said first puncture element and said suture holding element, and a biasing device operative to urge said first puncture element and said suture holding element from the deployed position back to the retracted position.

10. The suturing assembly according to claim 1, wherein said first puncture element or said suture holding element is formed with a lower rim for leaning on the distal end of said lumen.
